(19) United States
(12) Patent Application Publication
(10) Pub. No.: US 2026/0358334 A1
(43) Pub. Date: Nov. 12, 2026

(54) Diagnostic device

(71) Applicant: Septest Limited, Potters Bar (GB)

(72) Inventor: Alistair Richard Ian Wilson, Leeds (GB)

(73) Assignee: Septest Limited, Potters Bar (GB)

(21) Appl. No.: 19/121,185

(22) PCT Filed: Oct. 30, 2023
(86) PCT No.: PCT/EP2023/080277

§ 371 Date: Apr. 15, 2025

(30) Foreign Application Priority Data
Oct. 30, 2022 (GB) 2216036.0

(51) Int. Cl. G01N 33/543

(52) U.S. Cl. CPC G01N 33/54388; G01N 2333/01; G01N 2333/37; G01N 2333/4737; G01N 2333/585

(57) ABSTRACT

A test device for detecting the presence of analytes in a liquid sample, has a permeable material defining a first portion providing a first site for application of the liquid sample and for conjugates movably supported. Each conjugate comprises a binder for a respective analyte coupled to a respective detector reagent. A second portion is configured relative to the first portion so as to permit capillary flow communication therebetween. The second portion provides a second site spaced from the first site for visually determining the presence of the respective detector reagent, and comprising binders immobilized therein each of which binds to a respective analyte. The first portion comprises at least, for a first analyte, a first moveably supported conjugate comprising a first binder for a fungal biomarker substance coupled to a first respective detector reagent and, for at least one further said analyte, at least one further said moveably supported conjugate comprising at least one further binder coupled to at least one further respective detector reagent. The at least one further analyte comprises a respective biomarker substance selected from: a viral biomarker substance; a bacterial biomarker substance.

This application claims priority from GB2216036.0 filed 30 Oct. 2022, the contents and elements of which are herein incorporated by reference for all purposes.

### FIELD OF THE INVENTION

The present invention relates to diagnostic devices for diagnosis of liquid biological samples (e.g., blood) and particularly, although not exclusively, to devices for use in the diagnosis of sepsis.

### BACKGROUND

Sepsis is a potentially life-threatening inflammatory condition characterised by a dysregulated immune response to host tissue damage or infection by bacteria, fungi, viruses and, on rare occasions, parasites.

Symptoms of sepsis may include a fever, breathing difficulties, increased heart rate and mental confusion. If left untreated, sepsis can progress to septic shock characterised by dangerously low blood pressure and multiple organ dysfunction. It is estimated that currently 20% of all deaths worldwide are sepsis related.

A number of treatment options are available for the three most common types of pathogen induced sepsis; bacterial sepsis may be treated with antibiotics, viral sepsis with antivirals and fungal sepsis with antifungal agents. Epidemiological data varies, but bacterial sepsis may account for as much as 70% of all infective causes of sepsis.

Fungal infections account for approximately 15% of infections and are associated with some of the highest rates of inappropriate therapy and mortality (Candida species alone accounting for approximately 5% of all sepsis cases). Viruses account for only a small number of cases, approximately 1% to 2%, but data for viral cases is often not collected. The remaining causes being either mixed co-infections, or of unknown or potentially parasitic origin.

Hospitalized patients pose a much greater challenge to sepsis prevention because of their concurrent illness and an environment rich in pathogens. Currently, sepsis is initially screened for in healthcare settings through periodic clinical observations. In the UK, the National Early Warning Score (NEWS2) assessment system has been implemented by the UK's national healthcare service which includes analysis of respiratory rate, oxygen saturation, temperature, systolic blood pressure, pulse rate and any cognitive changes (similar screening and assessment protocols exist and are implemented around the world). Should a patient present with abnormal assessment results, sepsis may be suspected and blood samples taken for culture and laboratory evaluation.

Current blood-based laboratory tests used to aid sepsis diagnosis include blood gas (including glucose and lactate measurements), blood culture, full blood count, C-reactive protein (CRP), urea and electrolytes, creatinine and a clotting screen. Results from blood tests may take a couple of hours and in some cases, a few days from collection, depending on laboratory turnaround times and workflow arrangements.

Blood cultures are still seen as the ‘gold standard’ test for diagnosing sepsis and guiding treatment options, yet approximately 40% of positive blood culture results are false positives due to sample contamination. Furthermore, the results for blood culture tests can take 48 hours for bacterial cultures, with the results from fungal cultures taking as long as 72 hours. It has been suggested that in cases of bacterial sepsis, the chance of survival decreases by approximately 8% for every hour delay before treatment is given, with fungal cases requiring even more urgent treatment.

Therefore, a first priority in sepsis treatment and improved outcomes is early antimicrobial administration and source control. Current healthcare guidance states that a broad-spectrum antibiotic must be given at the maximum recommended dose without delay (ideally within 1 hour of identifying that the patient meets any high-risk criteria in an acute hospital setting), and not to wait for test results to confirm a bacterial infection. This approach is taken due to the fact that most sepsis cases are caused by a bacterial infection and because the effectiveness of any treatment for sepsis is time critical.

However, the use of broad-spectrum antibiotics in cases of non-bacterial sepsis can lead to additional complications, such as further opportunistic infections, potential antibiotic resistance and increased morbidity and mortality by delaying more appropriate targeted treatment. Furthermore, the use of unnecessary antibiotics for non-bacterial cases of sepsis is a significant drain on healthcare budgets. The clinical presentation of non-bacterial sepsis is not significantly different from that of bacterial sepsis. For example, fungal sepsis (with rare exceptions) does not present with specific clinical manifestations or laboratory abnormalities, meaning the identification and management of fungal sepsis remains a significant challenge.

To date, there is no single definitive biomarker for sepsis, that can replace the requirement for additional blood tests. Current point of care diagnostic tests are based on measuring the magnitude of an inflammatory response using non-specific inflammatory biomarkers and as such cannot reliably be used to distinguish between sepsis and other non-infectious critical illnesses, for example; Systemic Inflammatory Response Syndrome (SIRS), which presents with almost identical signs to sepsis. Furthermore, the costs and complexities associated with introducing such point of care test(s) has been a significant barrier to their wider adoption.

Therefore, the inventors have identified a need for a reliable, low cost, accessible rapid test device preferably capable of distinguishing between non-infectious critical illnesses and sepsis, which is preferably also capable of broadly identifying the trigger of sepsis as being bacterial, fungal or viral. This may be used to guide initial treatment and limit the overuse of antibiotics. The present invention has been devised in light of the above considerations.

### SUMMARY OF THE INVENTION

In a first aspect, the invention may provide a test device for detecting the presence of analytes in a liquid sample, the device comprising:
a permeable material defining:
a first portion providing a first site for application of the liquid sample and for conjugates movably supported therein wherein each conjugate comprises a binder for a respective analyte coupled to a respective detector reagent; and,
a second portion configured relative to the first portion so as to permit capillary flow communication therebetween, and the second portion providing a second site spaced from the first site for visually determining the presence of the respective detector reagent, and comprising binders immobilized therein each of which binds to a respective said analyte;
wherein the first portion comprises at least, for a first said analyte, a first said moveably supported conjugate comprising a first binder for a fungal biomarker substance coupled to a first detector reagent and, for at least one further said analyte, at least one further said moveably supported conjugate comprising at least one further binder coupled to at least one further detector reagent wherein the at least one further analyte comprises a respective biomarker substance selected from: a viral biomarker substance; a bacterial biomarker substance.

In this way, the test device may provide the combined, simultaneous testing, from the same liquid sample, for both a fungal biomarker and at least one of a viral biomarker and/or a bacterial biomarker.

By ‘biomarker’ it may be meant a chemical entity (or substance) that is capable of being measured to detect disease or other biochemical or physiological process and/or responses in a host organism. For example, a biomarker may comprise a naturally occurring molecule, gene, or characteristic by which a particular pathological or physiological process, disease, etc. can be identified. Examples include, but are not limited to: antigens; host derived antibodies; signalling molecules. The term “biomarker”, a portmanteau of “biological marker”, may refer to objective indications of medical state observed from outside the patient which can be measured accurately and reproducibly [see Kyle Strimbu and Jorge A. Tavel, M. D.: Curr Opin HIV AIDS. 2010 November; 5(6): 463-466].

The test device may comprise a further conjugate, and associated immobilized binder, for a non-specific inflammatory biomarker substance. Thus, a yet further analyte may comprise a non-specific inflammatory biomarker. The non-specific inflammatory biomarker substance may comprise C-Reactive Protein (CRP). Accordingly, the first portion may comprise a conjugate comprising a binder for a non-specific inflammatory biomarker substance comprising C-Reactive Protein (CRP) coupled to a yet further respective detector reagent, and the second portion may comprise a binder immobilized therein which binds to CRP.

Conjugates for biomarkers, and combinations thereof, may be employed in the test device. These may include conjugates for any one or more of: C-reactive protein (CRP), Procalcitonin (PCT), (1-3)-β-d-glucan (BDG), Myxovirus resistance protein A (MxA), Galactomannan (GM).

The combination of conjugates for biomarkers included in the test device may comprise conjugates for: BDG and PCT and MxA. Visual determination of three analytes (e.g., via three test lines) may be provided by use of three immobilized binders respectively for: BDG and PCT and MxA.

A combination of conjugates for biomarkers included in the test device may comprise conjugates for: BDG and PCT and MxA and CRP. Visual determination of four analytes (e.g., via four test lines) may be provided by use of four immobilized binders respectively for: BDG and PCT and MxA and CRP.

A combination of conjugates for biomarkers included in the test device may comprise conjugates for: GM and PCT and MxA. Visual determination of three analytes (e.g., via three test lines) may be provided by use of three immobilized binders respectively for: GM and PCT and MxA.

A combination of conjugates for biomarkers included in the test device may comprise conjugates for: GM and PCT and MxA and CRP. Visual determination of four analytes (e.g., via four test lines) may be provided by use of four immobilized binders respectively for: GM and PCT and MxA and CRP.

The first portion may comprise a porous pad configured for receiving the sample. When the liquid sample is (or comprises) a blood sample, the first portion may remove the red blood cells from the sample (preferably with minimal lysing) so as to enhance the visibility of an accumulation of the detector reagents associated with respective biomarkers.

For example, the second portion may comprise a porous pad or membrane (e.g., test pad or test membrane) configured for receiving the sample (or parts of the sample) delivered to it by the first portion.

The second porous pad may comprise a membrane of nitrocellulose or similar material. The second porous pad may comprise binders (e.g., immobilised test detection antibodies) for selected biomarkers immobilized at specified regions therein (e.g., to form test “capture lines”) where a user may view the results in the form of a visually detectable presence of the detector reagent (e.g., coloured particles) that have accumulated to form a concentration by being captured there.

A control site (e.g., a control line) may also be provided at the second portion which comprises a control binder immobilized therein which binds to a control conjugate provided in the first portion, wherein the control conjugate comprises a control detector reagent for visually determining the presence of the control detector reagent at the control site when the control conjugate is bound to the control binder there.

It is to be understood that the first portion may comprise two separate (but not separated) component parts. The two component parts may divide the first site into two sub-sites that are contiguous with each other or otherwise form a mutual contact supporting a capillary flow of liquid sample between them. A first component part of the first portion (e.g., a first of the two sub-sites) may comprise a porous pad for receiving the liquid sample and for supporting capillary fluid flow of the received sample to a second component part of the first portion (e.g., a second of the two sub-sites) that is in capillary fluid flow communication with the first component part and contains the conjugates movably supported therein. If the liquid sample comprises a blood sample, then the first component part may also serve to remove/filter red blood cells from the sample during capillary flow of the sample thorough it, such that the sample (or at least the remaining parts of it) delivered to the second component part is substantially free of red blood cells. The second component part is typically called a “conjugate pad” in the art, and this may hold the awaiting binders (e.g., binding molecules, such as antibody complexes or the like). Thus, application of the liquid sample may occur at a location upon the porous pad of the first sub-site that is laterally spaced from, and in flow communication with, the location of the conjugates in the porous pad of the second sub-site.

Alternatively, the first portion may comprise one continuous porous pad defining the whole of the first site as opposed to two separate component parts. In other words, one continuous porous pad may provide the site for application of the liquid sample and for conjugates movably supported therein. Of course, application of the liquid sample may occur at a location upon the one continuous porous pad that is laterally spaced from, and in flow communication with, the location of the conjugates.

Alternatively, each of the first portion and the second portion may comprise two (or more) separate sample flow channels collectively defining the first portion and the second portion of the device, wherein:
the first portion comprises collectively two respective channel first portions of the two sample flow channels (e.g., each formed of respective sample pads and respective conjugate pads etc.) collectively providing the first site (i.e., shared between two channels) for application of the liquid sample and for a respective conjugate(s) movably supported therein, and the respective conjugate(s) may comprise a binder(s) for a respective analyte(s) coupled to a respective detector reagent(s) (e.g., coloured particle(s)); and
the second portion comprises collectively two respective channel second portions of the two channels (e.g., each formed of a respective test zone pad) each being continuous with a respective one of the two channel first portions and collectively configured relative to a respective one of the two channel first portions so as to permit capillary flow communication therebetween. This collectively-defined second portion provides the second site in the collective form of two separate channel second sites (one per channel), each of which is spaced from the collectively-defined first site, for visually determining the presence of a respective detector reagent (e.g., coloured particle) associated with a respective one of the analytes. Each one of the two separate channel second sites comprises a binder(s) immobilized therein which binds to a respective one of the analytes. The resulting device structure provides that selected biomarker tests may be separated out into different channels. This may be desirable in order to increase or decrease the sensitivity of one of the tests, for example, by using different desensitising agents, sensitising agents etc. or to reduce interferences, such as non-specific binding or competition effects.

The test device may comprise a third portion configured relative to the second portion so as to permit capillary flow communication therebetween for receiving parts of the sample that have passed through the second portion and have not been bound to a binder within the second portion. In this way, the third portion may serve to mop up excess fluid at the end of a test.

Visually determining the presence of the detector reagent may be done by simple naked eye detection. Detector reagents most preferably comprise particles which, when accumulated in sufficient concentration, visibly display a predefined and recognisable colour. Here the term ‘particles’ may be understood to include a reference to a material in particulate form (e.g., nano-particles of a specified material) as well as a reference to biological particles such as particles (e.g., molecules) of an enzyme substance (e.g., protein molecules). Detector reagents may comprise coloured particles in this sense.

Various types of detector reagents can be used for the visualization of a signal. Examples readily available to the person of ordinary skill in the art include (but are not limited to):
coloured material in particulate form (e.g., latex beads, colloidal metal particles (e.g., gold), magnetic particles (e.g., permitting additional detection modes using magnetic sensors));
particles of a fluorescent material in particulate form (i.e., fluorescing with a pre-defined colour of fluorescent light);
enzyme conjugates

Test-specific binders (binding reagents) may include, without limitation: antibodies, antibody fragments, antigens, enzymes, aptamers or affimers. Any of these may be conjugated to a suitable detection reagent or molecular responder system such as (but not limited to) nanoparticles, nanoshells or enzyme linked systems for the selective binding and detection of each individual biomarker. Examples of suitable binders (e.g. antibodies, etc.) that may be used as detector and/or responder antibodies are (but not limited to) the following:
CRP Antibodies: Available from BBI Solutions; CRP Recombinant Fab Monoclonal Antibody BR228-D4A3. Available from Creative Biolabs; Anti-CRP Recombinant Antibody (clone E8-G1) (CAT #: VS3-WK63).
PCT Antibodies: Available from BBI Solutions; Anti-Procalcitonin BM448-V4A1, BM448-K8C7, BM448-H9F1.
BDG Antibodies: Available from Creative Biolabs; Recombinant Anti-1,3-Beta-glucan Antibody (CAT #: MOB-0228MC). Available from ThermoFisher Scientific; Fungal beta glucan Monoclonal Antibody (8201), Invitrogen™
MxA Antibodies: Available from NSJ Bioreagents; Myxovirus resistance protein 1 Antibody/MX1/MxA (RQ7192). Available from Sigma Aldrich; Anti-MxA, clone M143 (CL143).
GM Antibodies: Available from ThermoFisher Scientific; Aspergillus Monoclonal Antibody (5145). Available from Creative Biolabs; Mouse Anti-Galactomannan-2 Monoclonal Antibody (CGYJ217).

Preferably, the detector reagent, e.g., when a material in a particulate form, comprises a population of particles that is monodisperse (e.g., characterized by particles of uniform size in a dispersed phase). For example, particles of the detector reagent comprise a substantially spherical shape. In use, when a test is run using the test device, the particles are required to move through a torturous pore structure of the first and second portions of the test device. Particles (e.g., material in particulate form) may comprise an average diameter between 5 nm-150 nm, even more preferably still, a diameter between 20 nm-80 nm. The at least one further analyte may comprise only a viral biomarker substance, or only a bacterial biomarker substance. More preferably, the at least one further analyte may comprise both (i.e., two separate biomarkers present simultaneously) a viral biomarker substance, and a bacterial biomarker substance.

Accordingly, the test device may be used to detect a fungal biomarker and one more biomarker which could just be a viral biomarker (e.g., employing two test lines in total) or bacterial biomarker (e.g., employing two test lines in total) or two more biomarkers which could include a viral biomarker and a bacterial biomarker (e.g., employing three test lines in total). In other words, the test device may comprise two conjugates in total, the first being fungal and the second being either bacterial or viral, or may comprise three conjugates in total, the first being fungal, the second being bacterial and the third being viral. Indeed, the test device may comprise four conjugates in total, the first being fungal, the second being bacterial, the third being viral and the fourth being a conjugate for a non-specific inflammatory biomarker (e.g. CRP, as discussed below).

Most preferably, the colour of the detector reagents (e.g., coloured particles) is visually distinct from the colour of the second portion such that a concentration of immobilized detector reagents (e.g., coloured particles) at the second site is visually identifiable/determinable by the user as a concentration of a colour, in use, distinct from the colour of the parts of the second portion that surround (or are immediately adjacent to) the second site. The second site may be a linear section of the second portion, such that an accumulation of detector reagents (e.g., coloured particles) forms a visible coloured line upon the second portion. For example, the second site may be a test line defined by the linear section of the test pad or test membrane defined by the second portion. Preferably, test lines for respective analytes are separated from each other laterally along the second portion. The colour of any one of the detector reagents (e.g., coloured particles) may be the same as, or different from, the colour of any one of (or each of) the other detector reagents.

The test device may be employed at the point of need in any healthcare setting for patients who arrive presenting sepsis symptoms, allowing for more rapid admission and targeted treatment. The invention, in preferred aspects, may be implemented as a lateral flow device (LFD), also known as a lateral flow test (LFT), lateral flow immunoassay (LFIA) or lateral flow assay (LFA). This may provide a simple, low cost, rapid diagnostic solution for the initial diagnosis of a range of diseases.

Such a test need not (but could) replace the use of lab-based tests and cultures, rather, such a test could be used to establish a more rapid confirmation of the correct treatment course for the patient at a significantly lower cost than current test methods. This, may help in significantly increase survival rates, reduce hospital stay times, reduce the overall cost of treatment and enhance antibiotic stewardship measures.

Examples of nonspecific inflammatory biomarkers showing widely accepted correlation with sepsis cases include IL-6, IL-10 and CRP.

Procalcitonin (PCT) is capable of distinguishing bacterial sepsis from other non-infectious critical illness and to guide the use of antibiotic therapy. In healthy individuals, procalcitonin levels are nearly undetectable, rising significantly in a response to a pro-inflammatory stimulus, especially of bacterial origin. Furthermore, it does not rise significantly with viral or non-infectious inflammation.

Likewise, (1-3)-β-d-glucan (BDG) may be used to detect the presence of fungal infections. BDG is an attractive biomarker as it originates from the cell walls of a broad range of fungal agents, including the commonly encountered agents Candida spp., Aspergillus spp., and Pneumocystis jirovecii.

Current turnaround times for PCT blood tests in hospital laboratories is approximately 3 to 5 hours, which is a long time to wait in the context of sepsis diagnosis, and the turnaround time for BDG blood tests in some hospital laboratories is approximately 48 hours which is a dangerously long time in the context of sepsis diagnosis. In view of this, the invention may provide an improvement in how data obtainable from existing accepted biomarkers is made available quickly and easily at the actual point of need (e.g., at the bedside) to promptly guide earlier and more effective treatment.

The liquid sample may be (or comprise) a blood sample. For example, the sample may be a ‘raw’ and undiluted blood sample, or may be a liquid sample comprising blood and further comprising a diluent such as a test diluent, of pre-treatment. For example, a liquid sample may comprise a diluent comprising a test enhancing reagent(s) such as specific lysing agent(s) (e.g., this may be a preferred option when using for MxA, as discussed herein) or may comprise agglutinating agents, lysing agents which may be added to dilute a ‘raw’ blood sample prior to the sample being administered to the test device or while in the test device. For example, a user of the device one may wish to agglutinate the red blood cells in a diluent first, and/or lyse white blood cells to release any additional intracellular MxA in a diluent pot, and to use the resulting diluted blood sample ad the liquid sample to apply to the test device.

A concentration of an immobilized binder, for a given biomarker, within the second portion may be provided such that an accumulation of a detector reagent there is (e.g., becomes visible) visually determinable as bound to the immobilized binder (e.g., to form a visible test line result), or such that an accumulation of a detector reagent saturates, when a concentration of the given biomarker within the liquid sample has a pre-set threshold value. Preferably, the pre-set threshold value in question is set relative to an equivalent concentration of the biomarker in a corresponding sample of ‘raw’ undiluted blood. This allow users of the test device to employ test diluents, pre-treatments etc, without affecting the clinical concentrations they wish to use for diagnosis i.e., the test line concentration points are preferably set relative to the undiluted clinical threshold value for each biomarker in question. In other words, the pre-set threshold value may be considered to be a minimum sensitivity value for biomarker detection, in terms of concentration within the liquid sample, which is applicable to ‘raw’, undiluted blood samples having by definition a higher biomarker concentration than they would otherwise have if diluted by a diluent etc.

For example, if the liquid sample is intended to be a diluted blood sample comprising a diluent added to a ‘raw’ blood sample that dilutes the ‘raw’ blood sample by a pre-set dilution degree/amount, then the concentration of an immobilized binder, for a given biomarker, within the second portion may be provided such that an accumulation of a detector reagent there is (e.g., becomes visible) visually determinable as bound to the immobilized binder (e.g., to form a visible test line result), or such that an accumulation of a detector reagent saturates, when a concentration of the given biomarker within the diluted blood sample has a pre-set threshold value that corresponds directly to (i.e., is equivalent to, or is an objective proxy for) a pre-set concentration of the given biomarker within the ‘raw’, undiluted blood sample in accordance with the pre-set dilution degree/amount. In those circumstances, when the device is configured to be used on diluted blood samples the configuration of an immobilized binder (e.g., concentration of the binder, or sensitivity of the binder) for a given biomarker within the second portion (or the visibility of the detector reagent) may be adjusted (e.g., be higher) relative to what it would be the case when the test device is configured to be used on ‘raw’, undiluted samples, but nevertheless both configurations (‘raw’ use and ‘diluted’ use) are usable to detect the same pre-set concentration of biomarker within a liquid sample comprising blood that they are configured to diagnose.

In the test device, according to some embodiments of the invention, the fungal biomarker substance may comprise Beta-D-glucan (BDG).

The liquid sample may be (or comprise) a blood sample and the immobilized binder at the second portion may be configured such that an accumulation of the first detector reagent (e.g., coloured particles) is visually determinable as bound to the immobilized binder via the fungal biomarker comprising BDG, when a concentration of the fungal biomarker within the liquid sample is at least about 60 pg/mL. The inventors have found that this concentration of BDG biomarker in a liquid sample comprising blood (e.g., undiluted) results in a sufficient accumulation of immobilized detector reagent (e.g., coloured particles) within the second portion (i.e., having become bound at the binder there) to start to be reliably visually determined.

The concentration of the immobilized binder within the second portion may be chosen to be sufficient to achieve this result.

The liquid sample may be (or comprise) a blood sample and the immobilized binder at the second portion may be configured such that an accumulation of the first detector reagent (e.g., coloured particles) bound to the immobilized binder via the fungal biomarker comprising BDG saturates when a concentration of the fungal biomarker within the liquid sample is at least about 180 pg/mL. The inventors have found that this concentration of BDG biomarker in a liquid sample comprising blood (e.g., undiluted) results in a sufficient accumulation of immobilized detector reagent (e.g., coloured particles) within the second portion (i.e., having become bound at the binder there) to create a maximally visually determinable accumulation of detector reagent (e.g., coloured particles). The concentration of the immobilized binder within the second portion may be chosen to be sufficient to achieve this result.

In the test device, according to some embodiments of the invention, the fungal biomarker substance may comprise Galactomannan (GM).

The liquid sample may be (or comprise) a blood sample and the immobilized binder at the second portion may be configured such that an accumulation of the first detector reagent (e.g., coloured particles) is visually determinable as bound to the immobilized binder via the fungal biomarker comprising GM, when a concentration of the fungal biomarker within the liquid sample corresponds to an optical density index, ODI, value of at least about 0.5. The inventors have found that this concentration of GM biomarker in a liquid sample comprising blood (e.g., undiluted) results in a sufficient accumulation of immobilized detector reagent (e.g., coloured particles) within the second portion (i.e., having become bound at the binder there) to start to be reliably visually determined. The concentration of the immobilized binder within the second portion may be chosen to be sufficient to achieve this result.

The liquid sample may be (or comprise) a blood sample and the immobilized binder at the second portion may be configured such that an accumulation of the first detector reagent (e.g., coloured particles) bound to the immobilized binder via the fungal biomarker comprising GM saturates when a concentration of the fungal biomarker within the liquid sample corresponds to an optical density index, ODI, value of at least about 1.5. The inventors have found that this concentration of GM biomarker in a liquid sample comprising blood (e.g., undiluted) results in a sufficient accumulation of immobilized detector reagent (e.g., coloured particles) within the second portion (i.e., having become bound at the binder there) to create a maximally visually determinable accumulation of detector reagent (e.g., coloured particles). The concentration of the immobilized binder within the second portion may be chosen to be sufficient to achieve this result.

References herein to Optical Density, OD, may include a reference to optical Absorbance, as would be readily understood by the person of ordinary skill in the art.

The relative amount of light transmitted through a liquid sample may be defined as the ratio of the intensity of incident light, I₀, upon a sample, e.g., comprising GM, to the intensity of transmitted light, I, through the sample. This ratio may define a transmittance, T, of the analyte:

$$T = \frac{I}{{I}_{0}}$$

The absorbance, A, may be defined as:

$$A = log [ \frac{I}{{I}_{0}} ] = log [ T ]$$

The “optical density”, OD, may be defined as:

$$OD = log [ \frac{{I}_{0}}{I} ] = - log [ T ] = - log [ \frac{\% T}{100} ]$$

For absorbance measurements, the “optical density”, OD, may be so defined as a logarithmic measurement of the percent transmission (% T) in this way.

An optical density is preferably determined in respect of a particular wavelength of light corresponding to a peak, or maximum, absorbance value of the detector reagent within a spectral range of wavelengths of light of an absorbance spectrum of the detector reagent. For example, if gold nano-particles are used as detector reagent, then the spectral range of wavelengths may be from about 500 nm to about 575 nm, and the particular wavelength of light may be a value (e.g., one or 520 nm, 530 nm, 540 nm, 560 nm or 570 nm) corresponding to the spectral position of an absorbance peak/maximum therein. The spectral position of an absorbance peak/maximum may depend upon the properties (e.g., size, material) of the detector reagent used. For example, a similar condition is discussed in relation to, for example, a detector reagent comprising gold nano-particles (cf. FIG. 5 therein) in:

Manta et al.: “Optical Density Optimization of Malaria Pan Rapid Diagnostic Test Strips for Improved Test Zone Band Intensity” Diagnostics 2020, 10(11), 880; https://doi.org/10.3390/diagnostics10110880

The same principle applies to other forms of detector reagent.

The absorbance of the second site (e.g., a test line area) in the presence of an analyte, e.g., GM, may be related to a concentration of the analyte according to a pre-set relation, known in the art as a calibration curve, which defines a relationship between analyte concentration and optical density of the second site (e.g., a test line area). The optical density is preferably determined under a condition that a relation between the concentration of GM and the absorbance value being measured is a substantially linear relation. For example, a similar condition is discussed (cf. FIG. 1 therein) in:

Mercier et al.: “Galactomannan, a Surrogate Marker for Outcome in Invasive Aspergillosis: Finally Coming of Age”, Front. Microbiol., 4 Apr. 2018, Volume 9-2018, https://doi.org/10.3389/fmicb.2018.00661

Typically, Galactomannan tests are run using an immunoenzymatic sandwich microplate assay, such as for example, The Platelia™ Aspergillus EIA available from Bio-Rad. The test results from such assays are reported as ODI values and the GM clinical threshold values used throughout the literature, healthcare guidance and subsequently in this document are based on the ODI values established using this type of test. The assay works by first coating the wells of a microplate with monoclonal antibodies that are specific for Aspergillus galactomannan. The serum sample is then added to the wells and incubated. If Aspergillus galactomannan is present in the sample, it will bind to the antibodies on the microplate. Next, a conjugate reagent is added to the wells. This conjugate reagent is a mixture of monoclonal antibodies that are specific for Aspergillus galactomannan and that are linked to the enzyme peroxidase. The conjugate reagent binds to the Aspergillus galactomannan that is bound to the antibodies on the microplate. Finally, a substrate for peroxidase is added to the wells. This substrate reacts with the peroxidase in the conjugate reagent to produce a coloured product. The intensity of the colour is proportional to the amount of Aspergillus galactomannan that is present in the sample. An Optical Density (OD) value of the sample is then calculated by measuring the absorbance of the coloured product at 450 nm using a spectrophotometer.

The Optical Density, OD, value, as calculated by a spectrophotometer, is a measure of how much light is absorbed by a sample. The OD value is a measure of the concentration of a substance in a sample. The more concentrated a substance is, the more light it will absorb, and the higher the OD value will be. An Optical Density value of a control or cut-off sample (provided by the manufacturer) is then also calculated by measuring the absorbance of this coloured product at 450 nm using the spectrophotometer. The Optical Density Index (ODI) is then calculated thus:

ODI=Optical Density of test sample/Optical Density of cut-off sample

Test samples with an ODI<0.50 are considered to be negative for galactomannan antigen. Details can be found at in:

PLATELIA™ ASPERGILLUS EIA (96 TESTS 62796)—“THE PLATELIA™ ASPERGILLUS EIA IS AN IMMUNOENZYMATIC SANDWICH MICROPLATE ASSAY FOR THE DETECTION OF ASPERGILLUS GALACTOMANNAN ANTIGEN IN SERUM”, document code no. code: 881045, published by, and available from, Bio-Rad Laboratories Ltd, having an address at: The Junction, Station Road, Watford, Hertfordshire, WD17 1ET, United Kingdom.

See: https://commerce.bio-rad.com/webroot/web/pdf/inserts/CDG/en/62796_881045_EN.pdf

In the test device, according to some embodiments of the invention, the viral biomarker substance may comprise Myxovirus resistance protein A (MxA).

The liquid sample may be (or comprise) a blood sample and the immobilized binder at the second portion may be configured such that an accumulation of the at least one further detector reagent (e.g., coloured particles) is visually determinable as bound to the immobilized binder via the viral biomarker comprising MxA, when a concentration of the viral biomarker within the liquid sample is at least about 40 ng/mL. The inventors have found that this concentration of MxA biomarker in a liquid sample comprising blood (e.g., undiluted) results in a sufficient accumulation of immobilized detector reagent (e.g., coloured particles) within the second portion (i.e., having become bound at the binder there) to start to be reliably visually determined. The concentration of the immobilized binder within the second portion may be chosen to be sufficient to achieve this result.

The liquid sample may be (or comprise) a blood sample and the immobilized binder at the second portion may be configured such that an accumulation of the at least one further detector reagent (e.g., coloured particles) bound to the immobilized binder via the viral biomarker comprising MxA saturates when a concentration of the viral biomarker within the liquid sample is at least about 400 ng/mL. The inventors have found that this concentration of MxA biomarker in a liquid sample comprising blood (e.g., undiluted) results in a sufficient accumulation of immobilized detector reagent (e.g., coloured particles) within the second portion (i.e., having become bound at the binder there) to create a maximally visually determinable accumulation of detector reagent (e.g., coloured particles). The concentration of the immobilized binder within the second portion may be chosen to be sufficient to achieve this result.

The first portion may comprise a conjugate comprising a binder for a non-specific inflammatory biomarker substance comprising C-Reactive Protein (CRP) coupled to a yet further respective detector reagent, and the second portion may comprise a binder immobilized therein which binds to CRP. Thus, for example, the test device may comprise a fourth conjugate for a non-specific inflammatory biomarker substance. Thus, the at least one further analyte may comprise a non-specific inflammatory biomarker. In the test device, according to some embodiments of the invention, the non-specific inflammatory biomarker substance may comprise C-Reactive Protein (CRP).

The liquid sample may be (or comprise) a blood sample and the immobilized binder at the second portion may be configured such that an accumulation of the yet further detector reagent (e.g., coloured particles) is visually determinable as bound to the immobilized binder via the non-specific inflammatory biomarker comprising CRP, when a concentration of the non-specific inflammatory biomarker within the liquid sample is at least about 10 mg/L. The inventors have found that this concentration of CRP biomarker in a liquid sample comprising blood (e.g., undiluted) results in a sufficient accumulation of immobilized detector reagent (e.g., coloured particles) within the second portion (i.e., having become bound at the binder there) to start to be reliably visually determined. The concentration of the immobilized binder within the second portion may be chosen to be sufficient to achieve this result.

The liquid sample may be (or comprise) a blood sample and the immobilized binder at the second portion may be configured such that an accumulation of the yet further detector reagent (e.g., coloured particles) bound to the immobilized binder via the non-specific inflammatory biomarker comprising CRP saturates when a concentration of the non-specific inflammatory biomarker within the liquid sample is at least about 100 mg/L. The inventors have found that this concentration of CRP biomarker in a liquid sample comprising blood (e.g., undiluted) results in a sufficient accumulation of immobilized detector reagent (e.g., coloured particles) within the second portion (i.e., having become bound at the binder there) to create a maximally visually determinable accumulation of detector reagent (e.g., coloured particles). The concentration of the immobilized binder within the second portion may be chosen to be sufficient to achieve this result.

In the test device, according to some embodiments of the invention, the bacterial biomarker substance may comprise Procalcitonin (PCT).

The liquid sample may be (or comprise) a blood sample and the immobilized binder at the second portion may be configured such that an accumulation of the at least one further detector reagent (e.g., coloured particles) is visually determinable as bound to the immobilized binder via the bacterial biomarker comprising PCT, when a concentration of the bacterial biomarker within the liquid sample is at least about 0.5 ng/mL. The inventors have found that this concentration of PCT biomarker in a liquid sample comprising blood (e.g., undiluted) results in a sufficient accumulation of immobilized detector reagent (e.g., coloured particles) within the second portion (i.e., having become bound at the binder there) to start to be reliably visually determined. The concentration of the immobilized binder within the second portion may be chosen to be sufficient to achieve this result.

The liquid sample may be (or comprise) a blood sample and the immobilized binder at the second portion may be configured such that an accumulation of the at least one further detector reagent (e.g., coloured particles) bound to the immobilized binder via the bacterial biomarker comprising PCT saturates when a concentration of the bacterial biomarker within the liquid sample is at least about 5.0 ng/mL. The inventors have found that this concentration of PCT biomarker in a liquid sample comprising blood (e.g., undiluted) results in a sufficient accumulation of immobilized detector reagent (e.g., coloured particles) within the second portion (i.e., having become bound at the binder there) to create a maximally visually determinable accumulation of detector reagent (e.g., coloured particles). The concentration of the immobilized binder within the second portion may be chosen to be sufficient to achieve this result.

The test device may comprise conjugates and respective immobilized binders for at least BDG (fungal) or GM (fungal), PCT (bacterial) and MxA (viral). The test device may comprise three respective test lines (of immobilized binders) in the second portion accordingly. The test device may comprise conjugates and respective immobilized binders for at least BDG (fungal) or GM (fungal), PCT (bacterial) and MxA (viral) and CRP (non-specific inflammatory biomarker). The test device may comprise four respective test lines (of immobilized binders) in the second portion accordingly, the fourth test line (binding the non-specific inflammatory biomarker comprising CRP and its respectively co-attached conjugate) being useful to confirm that there is some inflammation going on.

Capture reagents (i.e., said binders) may be applied in a suitable medium to provide consistent application and functionality once bound. For example, a 0.1 Molar phosphate buffer at pH 7.4 may be used. For example, a compound of sodium phosphate dibasic heptahydrate at 0.07541 M may be used, or a compound of sodium phosphate monobasic monohydrate at 0.02459 M may be used. Other buffering systems could be utilised for example Tris based buffers and the inclusion of other specific agents can be utilised if needed, for example but not limited to bovine serum albumin, sugars or other agents to control non-specific binding or to improve reagent stability.

Preferably, only when the concentration of the biomarker in the sample exceeds its pre-set threshold value will a positive test line be visibly displayed (i.e. it ignores “normal” levels of each biomarker in the blood sample). This gives the user a “go/no-go” type result rather than a line intensity result. The ability to not require a separate test reader has certain advantages in the field of use such as cost reduction, zero calibration/training requirements and improved accessibility at the point of need.

Above the threshold levels the intensity of the test line preferably increases in proportion to the concentration of the biomarker in the sample, so it can also be used with a test reader should the user require some form of semi-quantitative understanding of the severity of infection (if present). But in the initial triage of sepsis diagnosis this is not critical as further blood tests, cultures etc. will still be used to refine and monitor treatment. The test device allows a user to focus on the immediate initial diagnosis of sepsis and what type (bacterial, fungal or viral) which is key to saving lives and improving outcomes.

In a second aspect, the invention may provide a test device as disclosed herein wherein the permeable material is planar and the first site is laterally spaced from the second site along the planar permeable material such that the test device provides a lateral flow test device. For example, a lateral flow test device may be provided for detecting elevated levels of CRP, PCT and BDG to aid the early determination of the trigger of the inflammatory response. For example, a lateral flow test device may be provided for detecting elevated levels of PCT, MxA and BDG and/or GM and optionally CRP, to aid the early determination of the trigger of the inflammatory response.

In a further aspect, the invention may provide a diagnostic device for the early identification of microbial infection types, in biological samples. The invention may relate to diagnostic devices which aid rapid diagnosis of infection and disease. Particularly, although not exclusively, the invention may relate to the early identification of the type of infection (bacterial, fungal or viral) triggering a dysregulated immune response in suspected sepsis cases. In a general aspect, the invention may provide a diagnostic device for the detection of both bacterial and fungal infections in humans.

In a further general aspect, the invention may provide a single diagnostic device for detection of elevated levels (or clinically significant levels) of Procalcitonin (PCT), C-reactive protein (CRP) and (1-3)-β-D-glucan (BDG) in a biological sample, including (but not limited to) blood, sweat, sputum, mucus, urine or saliva. A biological sample may also include serum or plasma.

In another, further general aspect the invention may provide the ability to rapidly identify possible sepsis cases in humans at the point of care.

Furthermore, the device may be configured to identify an elevated general inflammatory response (as indicated by elevated levels of CRP) as well as distinguish between bacterial sepsis (as indicated by elevated levels of PCT) and fungal sepsis (as indicated by elevated levels of BDG) and thereby also giving guidance to the possibility of viral infection. Furthermore, the device may be configured to identify a general inflammatory response and the possibility of a viral infection, as indicated by elevated levels of CRP (along with the patient's recent medical history) as well as detecting and distinguishing bacterial infections (as indicated by elevated levels of PCT) and fungal infections (as indicated by elevated levels of BDG).

In another general aspect, the invention may provide the detection of elevated levels of PCT, BDG and CRP using a lateral flow device (LFD). Such device may be either a sandwich immunoassay or competitive assay, with both methods being known to those skilled in the art. The device may implement inhibition assay such as is known to those skilled in the art. The test being either in a dipstick or cassette-type format (for example, enclosed in a plastic housing).

In another general aspect, the invention may provide a lateral flow device for the combined detection of C-Reactive Protein (CRP), Procalcitonin (PCT) and (1-3)-β-d-glucan (BDG) in biological samples.

The detection of the target biomarkers (PCT, CRP and BDG) may be conducted using the same lateral flow device, i.e. a multiplex lateral flow device. Said detection may be performed on either a single test channel or on two or more separate test channels within the same device, depending on the materials and reagents chosen and their relative sensitivities for each biomarker. Said test channels may share or have their own sample entry port for receiving the biological sample under investigation.

The test sample may be either unprepared (as extracted from its source) or prepared (mixed with reagents to enhance the stability and/or the performance/detectability of the sample during the test). Examples of sample and test enhancing reagents may include, but are not limited to, pH mediating buffers, diluents, viscosity control fluids, enzymes, antibodies, and agglutination agents. Further examples of sample and test enhancing reagents may include selective lysing agents, such as leukocyte membrane lysing agents.

Sample and test enhancing reagents may be employed either prior to sample application or in situ once the sample has been applied to the device.

In another general aspect, the invention may provide the use of antibodies which have been conjugated to nanoparticles for the selective binding and detection of each biomarker (CRP, PCT and BDG). Said nanoparticles providing the visual indication of the presence of each biomarker when captured at its specific test line. Examples of suitable nanoparticles include, but are not limited to, gold, carbon or latex.

The invention may provide the use of said binders (also known as binding agents) on a form comprising (but not limited to): antibodies, antibody fragments, antigens, enzymes, aptamers or affimers. The binders are preferably conjugated to a suitable detection reagent or molecular responder system such as (but not limited to): nanoparticles; nanoshells; enzyme linked systems. These may provide a selective binding and detection of each individual biomarker (e.g., CRP, PCT, BDG etc.). The detection reagent or molecular responder system may provide the visual indication of the presence of each biomarker when captured in the second portion of the test device e.g., at its specific test line. Examples of suitable nanoparticle detection reagents include, but are not limited to: gold, carbon or latex.

The test device may provide the ability to establish an individual minimum response or threshold concentration value for each biomarker. By doing this, the test may confirm the presence of a specific biomarker (e.g., by displaying a visible line at the relevant test line) if it is present in the sample above the threshold concentration value set for that particular biomarker on the test device. In this way, the test may be configured to ignore (or be visually unresponsive to) low level or clinically normal concentrations of biomarkers and can be tuned to only display clinically significant levels as required for each clinical application and/or sample type.

In a further aspect, the invention may provide that the intensity of each test line is proportional to the concentration of each biomarker in the sample, therefore the test is not only qualitative but also quantitative (if required). The quantitative accuracy of the test may be enhanced by use of a controlled sample volume, magnetic or fluorescent labelled antibody conjugates along with a suitable companion lateral flow device reader, mobile device app, or camera, or meter and applicable transformative data algorithms.

Potential designs and materials that may be used to create a suitable multiplexed lateral flow device such as, for example, a sandwich type assay, will be obvious to those skilled in the art. Generally speaking, such a device may comprise a number of distinct zones, the first of which may be a sample pad (or pads) which is/are designed to accept the test sample, filter any unwanted sample components (for example, in the case of a whole blood sample, removing red blood cells) and to wick the sample towards an adjoined conjugate pad.

The conjugate pad (or pads) typically may contain the key test reagents, such as, but not limited to, biomarker specific nanoparticle-antibody conjugates and the control line specific conjugates. It is here that the biomarkers (if present in the sample) bind to their specific nanoparticle-antibody conjugate to form a biomarker conjugate complex.

The conjugate pad (or pads) may contain the key test reagents, such as (but not limited to): biomarker specific nanoparticle-antibody conjugates (or other specific binding agent and detection system) and any control line specific conjugates (if required). Biomarkers (if present in the sample) may bind to their specific nanoparticle-antibody conjugates to form biomarker-conjugate complexes.

A test zone may be provided and may adjoin the conjugate pad which may comprise a suitable wicking media to draw the sample into this zone, such as, but not limited to, a nitrocellulose membrane. Distinct test lines for each biomarker may be defined in this region. Desirably, only a single set of antibodies (or other biomarker specific capture reagent or feature) are immobilised at each test line, thereby allowing for selective capture of the corresponding biomarker conjugate complex at the relevant designated test line. For example, at the first test line, optionally only the CRP conjugate complex is captured. At a second test line, optionally only the PCT conjugate complex is captured. At the third test line, optionally only the BDG conjugate complex is captured. At a control line, optionally only the control line conjugate is captured. In some examples, excess biomarker conjugates may be captured to create a visible control line.

The control line may be a separate line beyond the test lines and may provide a visual indication that the test has been conducted successfully (or not).

An absorbent pad may be included at the end of the device to ensure the sample is drawn to the very end of the device and to capture any excess sample once past the test zone.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1i shows a perspective view of an example lateral flow device embodiment.

FIG. 1ii shows a cross-sectional view of the same example device of FIG. 1i.

FIG. 1iii shows a perspective view of the internal test features of the example device of FIG. 1i.

FIGS. 2i, 2ii, 2iii, 2iv and, 2v show perspective views of example test results as seen through the test window of the example device of FIG. 1i.

FIG. 3 shows a perspective view of an example multi-channel lateral flow device embodiment.

FIG. 4 shows perspective view of an example modification to the device of FIG. 1i in which a fourth test line is provided as seen through the test window of the modified example device of FIG. 1i.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

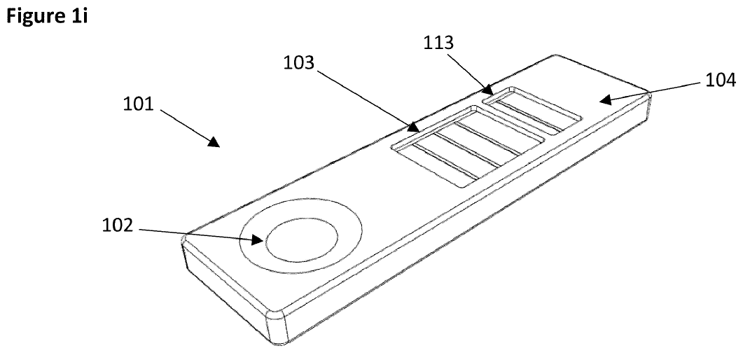

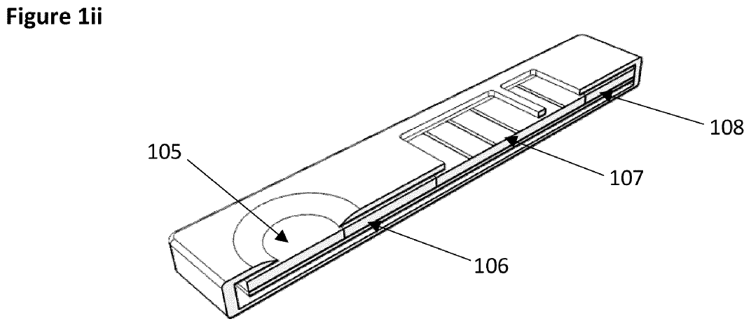

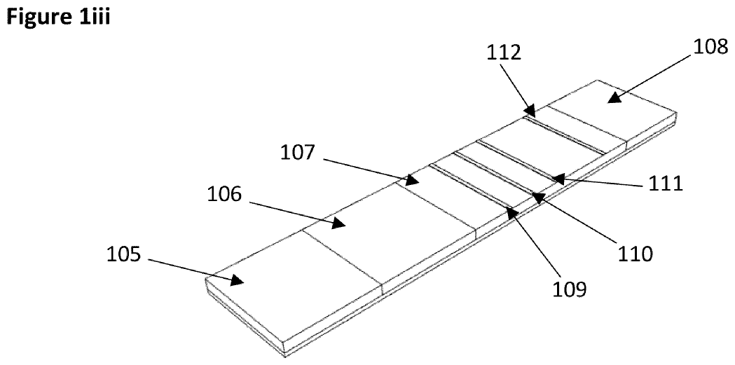

Referring to FIGS. 1i and 1ii, there is shown an example embodiment of the current invention in the form of a housed lateral flow device (101), which is unhoused in FIG. 1iii to show the internal test features and construction.

A test device (101) is configured as a lateral flow device for detecting the presence of analytes in a liquid sample. The device comprises a permeable material in the form of porous pads (105, 106, 107) defining: a first portion (105, 106) comprising a sample pad (105) and a conjugate pad (106) collectively configured for providing a first site for application of the liquid sample and for conjugates movably supported therein wherein each conjugate comprises a binder for a respective analyte coupled to a respective detector reagent; and, a second portion (107) defining a test zone and configured relative to the first portion (105, 106) so as to permit capillary flow communication therebetween. The second portion (107) provides a plurality of separate second sites (109, 110,111) each of which is spaced from the first site (105, 106) for visually determining the presence of the detector reagent (coloured particle). Each one of the second sites (109, 110, 111) comprises a binder immobilized therein which binds to a respective analyte.

The first portion comprises two separate component parts (two porous pads, 105, 106) that divide the first site into two contiguous sub-sites (105, 106) in mutual contact with each other to support a capillary flow of liquid sample between them. Application of the liquid sample occurs via a sample port (102) exposing a location upon the porous sample pad (105) forming a first sub-site that is laterally spaced from, and in flow communication with, the location of the conjugates in the porous conjugate pad (106) forming a second sub-site. The second component part (106) defines a so-called “conjugate pad” in that it holds the awaiting analyte binders (e.g., conjugates).

Thus, in this example, the first component part (105) of the first portion defines a first of the two sub-sites and comprises a porous sample pad for receiving the liquid sample and for supporting capillary fluid flow of the received sample to the second component part (106) of the first portion. The second component part forms a second one of the two sub-sites that is in capillary fluid flow communication with the first component part and contains the conjugates movably supported therein. When the liquid sample comprises a blood sample, the first component part (105) serves to remove/filter red blood cells from the sample during capillary flow of the sample thorough it. This means that the remaining parts of the sample delivered to the second component port (106) are substantially free of red blood cells.

In other examples (not shown), the first portion (105, 106) may comprise one continuous porous pad defining the whole of the first site for application of the liquid sample and for conjugates movably supported therein.

The first portion (105) comprises, for a first analyte, a first moveably supported conjugate comprising a first binder for a fungal biomarker substance coupled to a first coloured particle, and for an additional analyte, an additional moveably supported conjugate comprising a binder for a viral biomarker substance coupled to an additional coloured particle, and furthermore for a further analyte, a further moveably supported conjugate comprising a binder for a bacterial biomarker substance coupled to a further coloured particle. The first, additional and further detector reagents (coloured particles) may have the same colour, or may have different respective colours easily distinguishable from each other. The detector reagents (coloured particles) may have respective colours easily distinguishable from the colour of the second portion (107) where they are to be immobilized, bound and accumulated in use.

### Example 1

The Lateral flow device (101) consists of an outer housing (104) which may be made of a suitable polymeric material in a simple snap together configuration. At one end of the device and running through the top surface of the outer housing (104) is a sample port (102), which provides an access point for the sample to enter the device. The size and shape of the sample port (102) may be such that it helps to meter the volume of the sample that can be introduced. Directly below the sample port (102) is the sample pad (105) which serves to receive the sample and filter/hold back any unwanted sample components that may negatively affect the test (or how the test results are observed).

The material requirements for such a sample pad will depend on the sample type, but an example for whole blood samples may be (but not limited to) “Whatman 5” 2.5 μm filter paper, which can effectively retain red blood cells and prevent them from inhibiting the visual appraisal of the test results. Adjacent to and in direct contact and fluid communication with the sample pad (105) is the conjugate pad (106) which holds the test antibody conjugate reagents, including the control line conjugates. The concentrations of which are pre-determined based on the required clinical threshold values.

Adjacent to and in direct contact and fluid communication with the conjugate pad (106) is the test zone (107) made from a membrane of a suitable material known to those skilled in the art, for example (but not limited to) nitrocellulose (e.g., Sartorius CN95). The test zone (107) in this example has distinctive test lines (109, 110 and 111) which have specific individual capture antibodies immobilised on each line. For example (but not limited to), test line 1 (109) having specific immobilised capture antibodies ready to capture only CRP-conjugate complexes, test line 2 (110) having specific immobilised capture antibodies ready to capture only PCT-conjugate complexes and test line 3 (111) having specific immobilised capture antibodies ready to capture only BDG-conjugate complexes.

In some examples, the distinctive test lines (109, 110 and 111) may have specific individual capture reagents other than antibodies, such as for example (but not limited to) enzymes, antigens, aptamers or affimers immobilised on each line. For example (but not limited to), test line 1 (109) may have specific immobilised CRP capture reagents (such as, but not limited to, those previously disclosed) ready to capture only CRP-conjugate complexes, test line 2 (110) having specific immobilised PCT capture reagents (such as, but not limited to, those previously disclosed) ready to capture only PCT-conjugate complexes and test line 3 (111) having specific immobilised BDG capture reagents (such as, but not limited to, those previously disclosed).

The test zone (107) may be viewed through the test window (103). In this example, the control line (112), which captures only the control line specific conjugates, is positioned beyond and well away from the test lines (109, 110 and 111) and can be viewed through a separate control line window (113), but further embodiments may have test and controls lines viewable in the same test window. The control line (112) serves to verify that the test has run correctly i.e. that there was a suitable volume of sample to pass through the conjugate pad (106) and the test zone (107) to collect the reagents and perform the tests correctly. The control line (112) may serve to verify that the test has run correctly in that there was a suitable volume of and/or diluent liquid within the sample.

In some examples, separate control line specific conjugates may not be required, if there is an excess of biomarker conjugates, then these may be captured at the control line and used to display the validity of the test. This may be beneficial in certain circumstances to reduce the amount of non-specific binding interactions occurring at the test lines (109, 110 and 111).

Adjacent to and in direct contact and fluid communication with the test zone (107) is an absorbent pad (108) made from a suitable wicking material known to those skilled in the art, The absorbent pad (108) (e.g., Ahlstrom 222, or other material) serves to ensure the sample reaches the end of the device and collect any excess sample preventing it from leaking from the device.

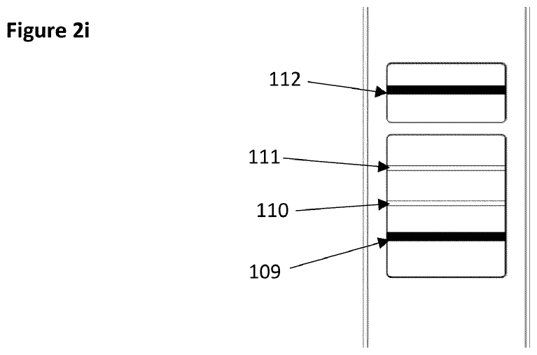

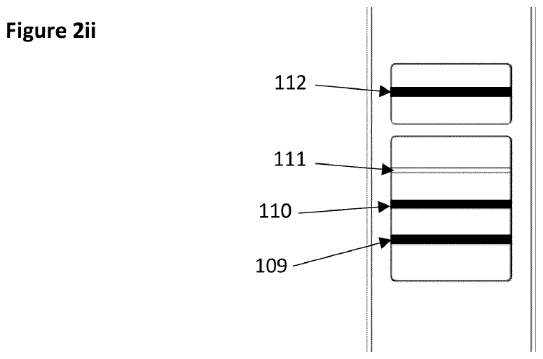

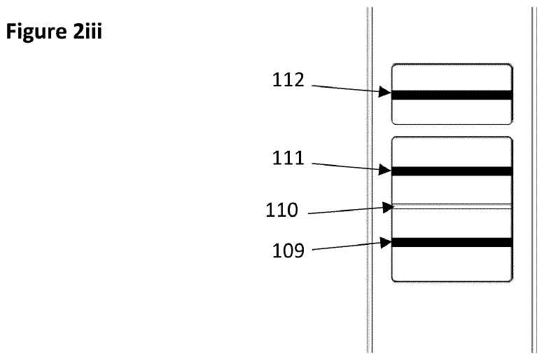

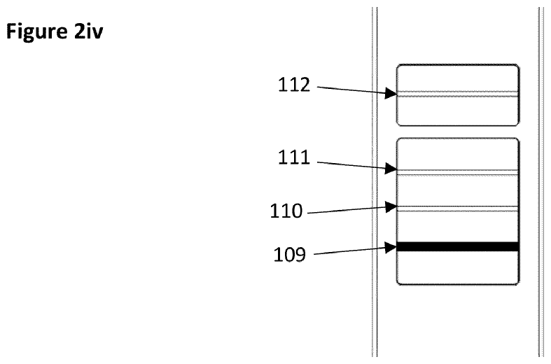

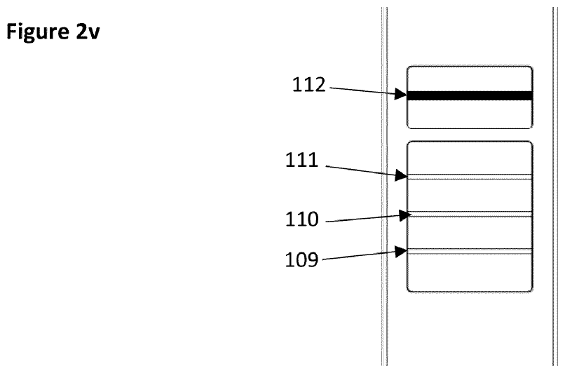

Referring to FIGS. 2i, 2ii, 2iii, 2iv and 2v, there is shown views of example test results as seen through the test and control windows (103 and 113 respectively) of the example device of FIG. 1i.

The following test line configurations will be used throughout the following descriptions in order to depict example potential test results that may be seen in the example embodiment of FIG. 1i.

Test line 1 (109) in this example will be specific to capturing the CRP-conjugate complex only.

Test line 2 (110) in this example will be specific to capturing the PCT-conjugate complex only.

Test line 3 (111) in this example will be specific to capturing the BDG-conjugate complex only.

Referring to FIG. 2i, test line 1 (109) is positive, as shown by a solid test line, suggesting an elevated level of CRP (e.g., indicative of general inflammation or possible infection). The elevated level may be a level above the pre-determined test response threshold concentration for CRP. Test line 2 (110) and test line 3 (111), (PCT and BDG respectively) are negative, as there is an absence of any visual solid test line, suggesting that bacterial or fungal infection is not detectable (therefore, viral sepsis may still be suspected). This may suggest that both PCT and BDG are not present in the sample to a clinically significant concentration and therefore a bacterial or fungal infection is not detectable (therefore, viral sepsis may still be suspected due to raised CRP level, unless ruled out by patient history or other clinical signs).

There is a solid visual control line (112) indicating that the test has run correctly and so is valid (e.g., the results are valid). Under these circumstances antibiotics may not be required.

Referring to FIG. 2ii, test line 1 (109) is positive, as shown by a visual solid test line, suggesting an elevated level of CRP (general inflammation). For example, this may suggest an elevated level of CRP is present in the sample above the pre-determined test response threshold concentration for CRP (indicative of general inflammation or infection). Test line 2 (110) is also positive, as shown by a visual solid test line, suggesting an elevated level of PCT (bacterial infection) and so under these circumstances antibiotics may be used. For example, this may suggest an elevated level of PCT is present in the sample above the test response threshold concentration set for PCT (indicative of a bacterial infection). Test line 3 (111) is negative, as there is an absence of any visual solid test line, suggesting that a fungal infection is not detectable, or is not present. This may suggest that BDG is not present in the sample above the test response threshold value set for BDG. There is a solid visual control line (112) indicating that the test has run correctly and so is valid (e.g., the results are valid). As there is an elevated level of PCT in this example test result, indicating a bacterial trigger, it is unlikely that the cause of sepsis is viral (or parasitic) unless the patient also presents with other concurrent viral (or parasitic) type symptoms (e.g., after further clinical evaluation of the patient and their case history).

Referring to FIG. 2iii, test line 1 (109) is positive, as shown by a visual solid test line, suggesting an elevated level of CRP (general inflammation). This may suggest an elevated level of CRP is present in the sample above the pre-determined test response threshold concentration for CRP (indicative of general inflammation or infection). Test line 2 (110) is negative, as there is an absence of any visual solid test line, suggesting that a bacterial infection is not detectable and so under these circumstances antibiotics should not be prescribed at this moment in time. This may suggest that PCT is not present in the sample above the test response threshold value set for PCT, as there is an absence of any visual solid test line. This may suggest that a bacterial infection is not present and so under these circumstances antibiotics should not be prescribed at this moment in time. Test line 3 (111) is positive, as shown by a visual solid test line, suggesting that a fungal infection is detectable. This may suggest an elevated level of BDG is present in the sample above the test response threshold concentration set for BDG (indicative of a fungal infection). There is a solid visual control line (112) indicating that the test has run correctly and so is valid (e.g., the results are valid). As there is an elevated level of BDG in this example test result, indicating a fungal trigger, it is (or may be) unlikely that the cause of sepsis is viral (or parasitic) unless the patient also presents with other concurrent viral (or parasitic) type symptoms (e.g., after further clinical evaluation of the patient and their case history).

Referring to FIG. 2iv, test line 1 (109) is positive, as shown by a visual solid test line, suggesting an elevated level of CRP (general inflammation). This may suggest an elevated level of CRP is present in the sample above the pre-determined test response threshold concentration for CRP (indicative of general inflammation or infection). Test line 2 (110) and test line 3 (111) are negative, as there is an absence of any visual solid test line. However, there is not a solid visual control line (112) which suggests that the test is not valid (i.e. the results are not to be trusted) and so a new device will need to be used and a new sample taken. This could be due to insufficient sample volume, incorrect sample preparation or a faulty test. In addition, this could be due to sample contamination or incorrect sample administration.

Referring to FIG. 2v, test line 1 (109), test line 2 (110) and test line 3 (111) are negative, as there is an absence of any visual solid test line. There is a solid visual control line (112) which suggests, or indicates, that the test is valid suggesting that there is insignificant general or infective inflammation present and so antibiotics may not be required. This may suggest that the levels of CRP, PCT and BDG in the sample are all under the clinically relevant test response threshold values, suggesting there is insignificant general or infective inflammation present and so antibiotics may not be required.

Of course, where all three lines and the control line are positive (not shown), this would indicate a co-infection.

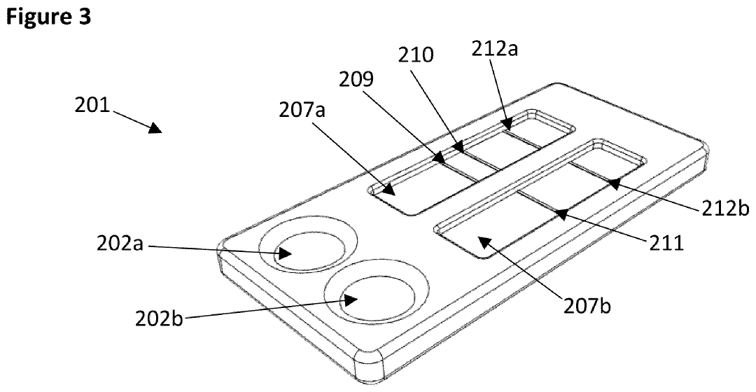

Referring to FIG. 3, there is shown an example embodiment of the current invention in the form of a multi-channel lateral flow device (201). In this embodiment the tests have been separated so, for example (but not limited to) CRP and PCT detection being run on the same test channel and BDG being run on a separate channel. This may be desirable in order to increase the sensitivity of one of the tests, for example, by using different sample concentrations, reagents, buffers, diluents etc. In particular, in this embodiment, certain biomarker tests have been separated out into different channels. This may be desirable in order to increase or decrease the sensitivity of one of the tests, for example, by using different desensitising agents, sensitising agents etc. or to reduce interferences, such as non-specific binding or competition effects.

The test device (201) is configured as a dual-channel lateral flow device for detecting the presence of analytes in a liquid sample. The device comprises a permeable material in the form of porous pads defining:
a first portion comprising two separate sample pads accessible via respective sample ports (202a, 202b) and two separate conjugate pads (not shown) collectively configured for providing a first site for application of the liquid sample and for respective conjugates movably supported therein. The conjugates comprise binders for respective analytes coupled to a coloured particle (which may be the same colour or different respective colours); and,
a second portion comprising two separate test zone pads visible via respective test channels (207a, 207b) each defining a respective part of a collective test zone which is configured relative to the two separate conjugate pads of the first portion so as to permit capillary flow communication therebetween.

The second portion provides a plurality of separate second sites (209, 210,211) each of which is spaced from the first site for visually determining the presence of the coloured particle associated with a respective one of the analytes. Each one of the second sites (209, 210, 211) comprises a binder immobilized therein which binds to a respective one of the analytes.

The multichannel lateral flow device (201) essentially operates in the same way as the previous embodiment (101) and uses the same general construction materials. However, this embodiment has two separate sample ports (202a and 202b) which are in direct contact and fluid communication with their respective separate conjugate pads and, test channels (207a and 207b respectively), along which the sample picks up the antibody conjugates and become captured by their respective immobilised capture antibodies at the relevant test line (209, 210 and 211). A separate control line (212a and 212b) is present on each test channel (207a and 207b respectively) to indicate the validity of each test channels performance.

### Example 2

Other conjugates for biomarkers, and combinations thereof, may be employed in the test device as discussed below. These include conjugates for: CRP, PCT, BDG, Myxovirus resistance protein A (MxA), Galactomannan (GM).

In preferred examples, the combination of conjugates for biomarkers included in the test device comprise conjugates for: BDG and PCT and MxA. For example, the three test lines 109, 110 and 111 of FIG. 1iii, or the three test lines 209, 210 and 211 of FIG. 3, may comprise binders respectively for: BDG and PCT and MxA. This is discussed in more detail in the example below.

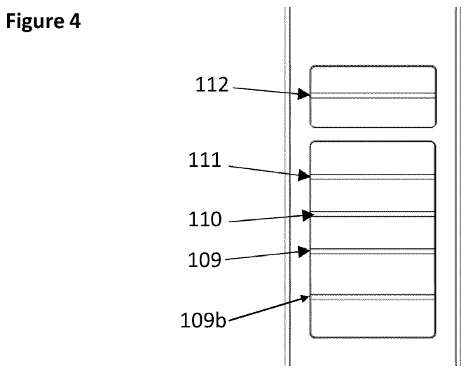

In other examples, the combination of conjugates for biomarkers included in the test device may comprise conjugates for: BDG and PCT and MxA and CRP. For example, the four test lines 109, 109b, 110 and 111, may be provided on the device, as shown in FIG. 4, which may comprise binders respectively for: BDG and PCT and MxA and CRP. This may be achieved simply by providing a fourth test line to the device described above with reference to FIGS. 1 to 2v. This is discussed in more detail in the example below.

In yet other examples, the combination of conjugates for biomarkers included in the test device may comprise conjugates for: Galactomannan (GM) and PCT and MxA. This is discussed in more detail in the example below. For example, the three test lines 109, 110 and 111 of FIG. 1iii, or the three test lines 209, 210 and 211 of FIG. 3, may comprise binders respectively for: Galactomannan (GM) and PCT and MxA. This is discussed in more detail in the example below.

In further examples, the combination of conjugates for biomarkers included in the test device may comprise conjugates for: Galactomannan (GM) and PCT and MxA and CRP. For example, the four test lines 109, 109b, 110 and 111, may be provided on the device, as shown in FIG. 4, which may comprise binders respectively for: Galactomannan (GM) and PCT and MxA and CRP. This may be achieved simply by providing a fourth test line to the device described above with reference to FIGS. 1 to 2v. This is discussed in more detail in the example below.

In other examples, referring to the figures, adjacent to and in direct contact and fluid communication with the sample pad (105) is the conjugate pad (106) which holds the test specific binding reagents, for example (but not limited to), antibodies, antibody fragments, antigens, enzymes, aptamers or affimers, which have been conjugated to a suitable detection reagent or molecular responder system such as (but not limited to) nanoparticles, nanoshells or enzyme linked systems for the selective binding and detection of each individual biomarker (CRP, PCT, MxA and BDG or GM). Preferably the nanoparticles will be either gold, carbon or latex. Even more preferably the nanoparticles will be gold nanoparticles with a diameter between 5 nm-150 nm, even more preferably still, gold nanoparticles with a diameter between 20 nm-80 nm.

The conjugate pad (106) may also hold the control line conjugates (if excess biomarker conjugates are not to be used at the control line). Such a conjugate pad may be made from, for example (but limited to), Ahlstrom 8980.

The following test line configurations will be used throughout the following descriptions in order to depict example potential test results that may be seen in the example embodiment of FIG. 1i, according to Example 2.

Test line 1 (109) in this example will be specific to capturing the BDG-conjugate complex only.

Test line 2 (110) in this example will be specific to capturing the PCT-conjugate complex only.

Test line 3 (111) in this example will be specific to capturing the MxA-conjugate complex only.

Referring to FIG. 2i, test line 1 (109) is positive, as shown by a solid test line, suggesting an elevated level of BDG (e.g., indicative of fungal infection). The elevated level may be a level above the pre-determined test response threshold concentration for BDG. Test line 2 (110) and test line 3 (111), (PCT and MxA respectively) are negative, as there is an absence of any visual solid test line, suggesting that bacterial or viral infection is not detectable (therefore, fungal sepsis may still be suspected). This may suggest that both PCT and MxA are not present in the sample to a clinically significant concentration and therefore a bacterial or viral infection is not detectable (therefore, fungal sepsis may still be suspected due to raised BDG level, unless ruled out by patient history or other clinical signs).

There is a solid visual control line (112) indicating that the test has run correctly and so is valid (e.g., the results are valid). Under these circumstances antibiotics may not be required.

Referring to FIG. 2ii, test line 1 (109) is positive, as shown by a visual solid test line, suggesting an elevated level of BDG (fungal infection). For example, this may suggest an elevated level of BDG is present in the sample above the pre-determined test response threshold concentration for BDG (indicative of fungal infection). Test line 2 (110) is also positive, as shown by a visual solid test line, suggesting an elevated level of PCT (bacterial infection) and so under these circumstances the use of antibiotics may be considered. For example, this may suggest an elevated level of PCT is present in the sample above the test response threshold concentration set for PCT (indicative of a bacterial infection). Test line 3 (111) is negative, as there is an absence of any visual solid test line, suggesting that a viral infection is not detectable, or is not present. This may suggest that MxA is not present in the sample above the test response threshold value set for MxA. There is a solid visual control line (112) indicating that the test has run correctly and so is valid (e.g., the results are valid). As there is an elevated level of BDG and PCT in this example test result, indicating a fungal and bacterial co-infection, the use of both antifungal and antibiotic treatments may be considered.

Referring to FIG. 2iii, test line 1 (109) is positive, as shown by a visual solid test line, suggesting an elevated level of BDG (fungal biomarker). This may suggest an elevated level of BDG is present in the sample above the pre-determined test response threshold concentration for BDG (indicative fungal infection). Test line 2 (110) is negative, as there is an absence of any visual solid test line, suggesting that a bacterial infection is not detectable. This may suggest that PCT is not present in the sample above the test response threshold value set for PCT, as there is an absence of any visual solid test line. This may suggest that a bacterial infection is not present and so under these circumstances antibiotics should not be prescribed at this moment in time. Test line 3 (111) is positive, as shown by a visual solid test line, suggesting that a viral infection is detectable. This may suggest an elevated level of MxA is present in the sample above the test response threshold concentration set for MxA (indicative of a viral infection). There is a solid visual control line (112) indicating that the test has run correctly and so is valid (e.g., the results are valid). As there is an elevated level of BDG and MxA in this example test result, indicating a fungal and viral co-infection, the use of both antifungal and antiviral treatments may be considered.

Referring to FIG. 2iv, test line 1 (109) is positive, as shown by a visual solid test line, suggesting an elevated level of BDG (fungal biomarker). This may suggest an elevated level of BDG is present in the sample above the pre-determined test response threshold concentration for BDG (indicative of fungal infection). Test line 2 (110) and test line 3 (111) are negative, as there is an absence of any visual solid test line. However, there is not a solid visual control line (112) which suggests that the test is not valid (i.e. the results are not to be trusted) and so a new device will need to be used and a new sample taken. This could be due to insufficient sample volume, incorrect sample preparation or a faulty test. In addition, this could be due to sample contamination or incorrect sample administration.

Referring to FIG. 2v, test line 1 (109), test line 2 (110) and test line 3 (111) are negative, as there is an absence of any visual solid test line. There is a solid visual control line (112) which suggests, or indicates, that the test is valid suggesting that there is insignificant infection present and so antibiotics may not be required. This may suggest that the levels of MxA, PCT and BDG in the sample are all under the clinically relevant test response threshold values, suggesting there is insignificant infection present and so antibiotics (and/or other antimicrobials) may not be required.

The concentrations of binding conjugates and immobilized binders may be pre-determined based on the required clinical threshold values for each specific biomarker (CRP, PCT, MxA, GM, BDG, etc.). Furthermore, the concentration and/or sensitivity of the test reagents (e.g., conjugates, binders and detector reagents) may be finely tuned such that the test is capable of essentially ignoring the presence of the individual biomarkers (CRP, PCT, MxA, GM, BDG, etc.) when present in the sample under a pre-determined or clinically significant concentration for each biomarker. In this way the test may only display (e.g. visually display) the presence of a biomarker if it is available in the sample to clinically relevant or elevated levels (i.e. above its individual threshold or response concentration). In this way the test can be tuned to both the application and the sample type.

In the case of CRP detection in a liquid sample comprising blood, the test threshold may be set to, for example (but not limited to), at least about 10 mg/L (the upper clinical threshold for normal levels of CRP in human blood samples). If CRP is present in the sample below this threshold concentration the test preferably will not display a line at the CRP test result line (i.e. a negative test result for CRP). Above this threshold concentration the test preferably will show a visible test line at the CRP test result line (i.e. a positive test result for CRP). The concentration of the immobilized binder may be chosen to be sufficient to achieve this result. The binder may be configured such that an accumulation of the detector reagent (e.g., coloured particles) for CRP saturates when a concentration of CRP within the liquid sample comprising blood is at least about 100 mg/L. The concentration of the immobilized binder may be chosen to be sufficient to achieve this result.

In the case of PCT detection in a liquid sample comprising blood, the test threshold may be set to, for example (but not limited to), be at least about 0.5 ng/mL (the upper clinical threshold for normal levels of PCT in human blood samples). If PCT is present in the sample below this threshold concentration the test preferably will not display a line at the PCT test result line (i.e. a negative test result for PCT). Above this threshold concentration the test preferably will show a visible test line at the PCT test result line (i.e. a positive test result for PCT). The concentration of the immobilized binder may be chosen to be sufficient to achieve this result. The binder may be configured such that an accumulation of the detector reagent (e.g., coloured particles) for PCT saturates when a concentration of PCT within the liquid sample comprising blood is at least about 5.0 ng/mL. The concentration of the immobilized binder may be chosen to be sufficient to achieve this result.

In the case of BDG detection in a liquid sample comprising blood, the test threshold may be set to, for example (but not limited to), at least about 60 pg/mL (the upper clinical threshold for normal levels of BDG in human blood samples). If BDG is present in the sample below this threshold concentration the test preferably will not display a line at the BDG test result line (i.e. a negative test result for BDG). Above this threshold concentration the test preferably will show a visible (positive) test line at the BDG test result line (i.e. a positive test result for BDG). An accumulation of the detector reagent (e.g., coloured particles) for BDG may be configured to saturate when a concentration of BDG within the liquid sample comprising blood is at least about 180 pg/mL. The concentration of the immobilized binder may be chosen to be sufficient to achieve this result.

In the case of Galactomannan (GM), the test threshold may be set such that an accumulation of detector reagent (e.g., coloured particles) may be visually determinable at the test line as bound to the immobilized binder via the GM biomarker when a concentration of GM within the liquid sample comprising blood corresponds to an optical density index, ODI, value of at least about 0.5. This concentration of GM biomarker in a liquid sample comprising blood (e.g., undiluted blood sample) results in a sufficient accumulation of immobilized detector reagent (e.g., coloured particles) to start to be reliably visually determined. Above this threshold concentration the test preferably will show a visible test line at the GM test result line (i.e. a positive test result for GM). The concentration of the immobilized binder may be chosen to be sufficient to achieve this result. The binder may be configured such that an accumulation of the detector reagent (e.g., coloured particles) for GM saturates when a concentration of GM within the liquid sample corresponds to an optical density index, ODI, value of at least about 1.5. The concentration of the immobilized binder may be chosen to be sufficient to achieve this result.

In the case of Myxovirus resistance protein A (MxA) detection in a liquid sample comprising blood, the test threshold may be set such that an accumulation of detector reagent (e.g., coloured particles) may be visually determinable at the test line as bound to the immobilized binder via the MxA biomarker when a concentration of biomarker within the liquid sample is at least about 40 ng/mL. Above this threshold concentration the test preferably will show a visible test line at the MxA test result line (i.e. a positive test result for MxA). The concentration of the immobilized binder may be chosen to be sufficient to achieve this result. The binder may be configured such that an accumulation of the detector reagent (e.g., coloured particles) for MxA saturates when a concentration of MxA within the liquid sample is at least about 400 ng/mL. The concentration of the immobilized binder may be chosen to be sufficient to achieve this result.

Additionally, selective and specific desensitisation may be achieved through the use of free (unconjugated) biomarker binding reagents or “blockers” such as for example, but not limited to, free (unconjugated), antibodies, antibody fragments, antigens, enzymes, aptamers, affimers or other desensitising, or direct, or in-direct competing agents.

Examples of suitable binders (e.g., antibodies) that may be used as detector and/or responder antibodies for PCT, CRP, MxA, GM and BDG are (but not limited to) the following:
CRP Antibodies: Available from BBI Solutions; CRP Recombinant Fab Monoclonal Antibody BR228-D4A3. Available from Creative Biolabs; Anti-CRP Recombinant Antibody (clone E8-G1) (CAT #: VS3-WK63).
PCT Antibodies: Available from BBI Solutions; Anti-Procalcitonin BM448-V4A1, BM448-K8C7, BM448-H9F1.
BDG Antibodies: Available from Creative Biolabs; Recombinant Anti-1,3-Beta-glucan Antibody (CAT #: MOB-0228MC). Available from ThermoFisher Scientific; Fungal beta glucan Monoclonal Antibody (8201), Invitrogen™
MxA Antibodies: Available from NSJ Bioreagents; Myxovirus resistance protein 1 Antibody/MX1/MxA (RQ7192). Available from Sigma Aldrich; Anti-MxA, clone M143 (CL143).
GM Antibodies: Available from ThermoFisher Scientific; Aspergillus Monoclonal Antibody (5145). Available from Creative Biolabs; Mouse Anti-Galactomannan-2 Monoclonal Antibody (CGYJ217).

The final choice of antibodies for each biomarker will depend on the combination of other reagents used in the test and the sensitivities required.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### REFERENCES

A number of publications are cited above in order to more fully describe and disclose the invention and the state of the art to which the invention pertains. Full citations for these references are provided below. The entirety of each of these references is incorporated herein.
[1] Kyle Strimbu and Jorge A. Tavel, M. D.: Curr Opin HIV AIDS. 2010 November; 5(6): pp 463-466.
[2] Manta et al.: “Optical Density Optimization of Malaria Pan Rapid Diagnostic Test Strips for Improved Test Zone Band Intensity” Diagnostics 2020, 10(11), p 880. https://doi.org/10.3390/diagnostics10110880
[3] Mercier et al.: “Galactomannan, a Surrogate Marker for Outcome in Invasive Aspergillosis: Finally Coming of Age”, Front. Microbiol., 4 Apr. 2018, Volume 9-2018. https://doi.org/10.3389/fmicb.2018.00661
[4] PLATELIA™ ASPERGILLUS EIA (96 TESTS 62796)—“THE PLATELIA™ ASPERGILLUS EIA IS AN IMMUNOENZYMATIC SANDWICH MICROPLATE ASSAY FOR THE DETECTION OF ASPERGILLUS GALACTOMANNAN ANTIGEN IN SERUM”, document code no. code: 881045, published by, and available from, Bio-Rad Laboratories Ltd, having an address at: The Junction, Station Road, Watford, Hertfordshire, WD17 1ET, United Kingdom.
   See: https://commerce.bio-rad.com/webroot/web/pdf/inserts/CDG/en/62796-881045 EN.pdf

1. A test device for detecting the presence of analytes in a liquid sample, the device comprising: a permeable material defining:
a first portion providing a first site for application of the liquid sample and for conjugates movably supported therein wherein each conjugate comprises a binder for a respective analyte coupled to a respective detector reagent; and,
a second portion configured relative to the first portion so as to permit capillary flow communication therebetween, and said second portion providing a second site spaced from the first site for visually determining the presence of the respective detector reagent, and comprising binders immobilized therein each of which binds to a respective said analyte;
wherein the first portion comprises at least, for a first said analyte, a first said moveably supported conjugate comprising a first binder for a fungal biomarker substance coupled to a first respective detector reagent and, for at least one further said analyte, at least one further said moveably supported conjugate comprising at least one further binder coupled to at least one further respective detector reagent wherein the at least one further analyte comprises a respective biomarker substance selected from: a viral biomarker substance; a bacterial biomarker substance.

2. A test device according to claim 1 wherein said fungal biomarker substance comprises Beta-D-glucan (BDG).

3. A test device according to claim 2 wherein the liquid sample comprises a blood sample and the immobilized binder at the second portion is configured such that an accumulation of the first detector reagent is visually determinable as bound to the immobilized binder via the fungal biomarker when a concentration of the fungal biomarker within the liquid sample is at least about 60 pg/mL.

4. A test device according to claim 2 wherein the liquid sample comprises a blood sample and the immobilized binder at the second portion is configured such that an accumulation of the first detector reagent bound to the immobilized binder via the fungal biomarker saturates when a concentration of the fungal biomarker within the liquid sample is at least about 180 pg/mL.

5. A test device according to claim 1 wherein said fungal biomarker substance comprises Galactomannan (GM).

6. A test device according to claim 5 wherein the liquid sample comprises a blood sample and the immobilized binder at the second portion is configured such that an accumulation of the first detector reagent is visually determinable as bound to the immobilized binder via the fungal biomarker when a concentration of the fungal biomarker within the liquid sample corresponds to an optical density index, GDI, value of at least about 0.5.

7. A test device according to claim 5 wherein the liquid sample comprises a blood sample and the immobilized binder at the second portion is configured such that an accumulation of the first detector reagent bound to the immobilized binder via the fungal biomarker saturates when a concentration of the fungal biomarker within the liquid sample corresponds to an optical density index, ODI, value of at least about 1.5.

8. A test device according to claim 1 wherein said viral biomarker substance comprises Myxovirus resistance protein A (MxA).

9. A test device according to claim 8 wherein the liquid sample comprises a blood sample and the immobilized binder at the second portion is configured such that an accumulation of the at least one further detector reagent is visually determinable as bound to the immobilized binder via the viral biomarker when a concentration of the viral biomarker within the liquid sample is at least about 40 ng/mL.

10. A test device according to claim 8 wherein the liquid sample comprises a blood sample and the immobilized binder at the second portion is configured such that an accumulation of the at least one further detector reagent bound to the immobilized binder via the viral biomarker saturates when a concentration of the viral biomarker within the liquid sample is at least about 400 ng/mL.

11. A test device according to claim 1 wherein said first portion comprises a conjugate comprising a binder for a non-specific inflammatory biomarker substance comprising C-Reactive Protein (CRP) coupled to a yet further respective detector reagent, and the second portion comprises a binder immobilized therein which binds to CRP.

12. A test device according to claim 11 wherein the liquid sample comprises a blood sample and the immobilized binder at the second portion is configured such that an accumulation of the yet further detector reagent is visually determinable as bound to the immobilized binder via the non-specific inflammatory biomarker when a concentration of the non-specific inflammatory biomarker within the liquid sample is o at least about 10 mg/L.

13. A test device according to claim 11 wherein the liquid sample comprises a blood sample and the immobilized binder at the second portion is configured such that an accumulation of the yet further detector reagent bound to the immobilized binder via the non-specific inflammatory biomarker saturates when a concentration of the non-specific inflammatory biomarker within the liquid sample is at least about 100 mg/L.

14. A test device according to claim 1 wherein said bacterial biomarker substance comprises Procalcitonin (PCT).

15. A test device according to claim 14 wherein the liquid sample comprises a blood sample and the immobilized binder at the second portion is configured such that an accumulation of the at least one further detector reagent is visually determinable as bound to the immobilized binder via the bacterial biomarker when a concentration of the bacterial biomarker within the liquid sample is at least about 0.5 ng/mL.

16. A test device according to claim 14 wherein the liquid sample comprises a blood sample and the immobilized binder at the second portion is configured such that an accumulation of the at least one further detector reagent bound to the immobilized binder via the bacterial biomarker saturates when a concentration of the bacterial biomarker within the liquid sample is at least about 5.0 ng/mL.

17. A test device according to claim 1 wherein the permeable material is planar and the second site is laterally spaced from the first site along the planar permeable material such that the test device provides a lateral flow test device.
